(19) United States
(12) Patent Application Publication
(10) Pub. No.: US 2019/0223761 A1
(43) Pub. Date: Jul. 25, 2019

(54) METHOD AND APPARATUS FOR QUANTIFYING LUNG FUNCTION

(71) Applicant: Oxford University Innovation Limited, Oxford (GB)

(72) Inventors: Peter Alistair ROBBINS, Oxford, Oxfordshire (GB); Gus HANCOCK, Oxford, Oxfordshire (GB); Grant RITCHIE, Oxford, Oxfordshire (GB); James MOUNTAIN, Oxford, Oxfordshire (GB); David O'NEILL, Oxford, Oxfordshire (GB); Jonathan WHITELEY, Oxford, Oxfordshire (GB); Luca CIAFFONI, Oxford, Oxfordshire (GB); John COUPER, Reading, Berkshire (GB)

(21) Appl. No.: 16/328,773

(22) PCT Filed: Aug. 15, 2017
(86) PCT No.: PCT/GB2017/052397

§ 371 Date: Feb. 27, 2019

(30) Foreign Application Priority Data
Sep. 1, 2016 (GB) 1614842.1
Sep. 8, 2016 (GB) 1615277.9

(51) Int. Cl. A61B 5/091; A61B 5/083; A61B 5/087; A61B 5/085

(52) U.S. Cl. CPC A61B 5/091; A61B 5/0833; A61B 5/085; A61B 5/087; A61B 5/0836

(57) ABSTRACT

A method and apparatus for non-invasive assessment of lung inhomogeneity by accurate high temporal resolution measurement of respiratory gas flows at the mouth during steady state breathing and inert gas wash-in or wash-out and using these measurements to fit a mathematical model of the inhomogeneous lung. The model of the lung is based on modelling the lung as plural alveolar compartments each having an identical volume at functional residual capacity, but varying in its fractional share of total lung compliance, total pulmonary vascular conductance and total anatomical deadspace. A bivariate log-normal distribution of the lung compliance and pulmonary vascular conductance is used, together with a normal distribution of deadspace fraction. The model is fitted to the measurements using non-linear regression and the distribution of ventilation: perfusion ratios, lung compliance: volume ratios, lung vascular conductance: volume ratios and lung deadspace: volume ratios obtained from the fitted model are indicative of the airway condition and thus lung function of the subject.

The present invention relates to a method and apparatus for quantifying lung function.

The quantification of lung function by pulmonary function tests (PFT) is important in the assessment of airways disease such as chronic obstructive pulmonary disease (COPD), asthma and cystic fibrosis. The quantification of lung function helps with assessing the degree and progress of disease and can be used by pharmaceutical companies to identify groups of patients for testing of different therapies and to assess the effectiveness of potential therapies.

The current gold standard pulmonary function test is the forced expiratory volume (FEV1) which is a measurement of the maximal amount of air that a patient can forcefully exhale in one second. This is the main pulmonary function test used to test for, and stage, COPD because it is inexpensive, quick and relatively simple to measure, and it integrates a number of features of COPD into a single measured variable. However, FEV1 does not detect early airway disease and is an extremely insensitive marker for changes in airway function. Its lack of sensitivity means that when used in Phase II clinical trials to assess the effectiveness of potential treatments, large numbers of patients must be recruited and a very high degree of patient cooperation is required.

Further, COPD is very heterogeneous. There are a number of distinct pathological changes that occur in the lung, their relative contributions to disease differ in different patients, and the degree of pathology varies significantly between different sections of the lung. These differences are reflected in the considerable variations in gas exchange within the diseased lung. The rate of decline in lung function also varies considerably between patients. Because COPD is so heterogeneous, there is considerable interest in profiling patients with COPD with a view to identifying sub-groups that may be amenable to different treatments.

Other techniques for lung function testing apart from spirometry, of which FEV1 is an example, are limited to measuring flows and volumes of particular types (addressing obstructive and restrictive patterns of lung disease), measuring carbon monoxide diffusing capacity (which measures the diffusing capacity of the lung) and measuring arterial blood gas. Other niche developments such as oscillometry or wash-out tests to give a lung clearance index have to date provided only limited information about the lung. For example, multiple-breath and single-breath inert gas wash-out tests (MBW and SBW, respectively) assess the efficiency of ventilation distribution in the lung. The paper “Ventilation-Perfusion Distribution in Normal Subjects” by Kenneth C Beck et al. (J. Appl. Physiol. 113: 872-877, 2012) discusses a wash-in experiment for examining the ventilation-perfusion distribution of the lung in normal subjects. The paper “Slow and Fast Lung Compartments in Cystic Fibrosis Measured by Nitrogen Multiple-Breath Wash-out” by Gustafsson et al. (J. Appl. Physiol. 117: 720-729, 2014) discusses a nitrogen MBW test for assessing the influence of airway disease on the uniformity of ventilation distribution in the lungs. The ERS/ATS consensus statement for inert gas wash-out measurement using multiple- and single-breath tests by P D Robinson et al. (European Respiratory Journal 41: 507-522, 2013) notes that inert gas wash-out tests using the single- or multiple-breath wash-out technique have been known for several decades and in giving measures of ventilation distribution inhomogeneity offer complimentary information to standard lung function tests and improved sensitivity in the detection of early lung damage. However it also notes that there are significant limitations and difficulties in current techniques.

According to the present invention there is provided a method of quantifying lung function of a subject comprising the steps of:
a. obtaining plural measurements of the amounts at the mouth of plural respiratory gases in inhaled and exhaled breath as a function of time within each breath of a plurality of successive breaths during a period of steady breathing and a period of inert gas wash-in or wash-out of a respiring subject;
b. outputting the measured amounts to a data processor adapted to model lung function by using a parameterised lung model adapted to predict expired amounts of the plural respiratory gases at the mouth of a respiring subject at the time point of each of said plural measurements;
c. varying the parameters of the lung model to fit the predicted expired respiratory gas amounts to the plural measurements; and
d. outputting at least one parameter of the lung model as a quantifier of lung function.

By using breath sampling within each breath across multiple breaths the pattern by which respiratory gas comes out during a single breath can be joined with information contained within the pattern by which respiratory gas comes out during multiple breaths (with different volumes, durations and inspirates) in determining the parameters of the model. This provides much more sensitive and discriminatory measurements of lung function than is currently available.

By steady breathing is meant the supply of a constant, stable composition of oxygen and carbon dioxide in the inspiratory gas (such as air) which will result in stable respiration and a relatively stable composition for oxygen and carbon dioxide in mixed expired gas (once the subject has settled down). Optionally a subject may need to be allowed to breathe steadily for 2 or 3 minutes to stabilize their breathing (some subjects hyperventilate initially in respiratory testing).

The respiratory gases preferably comprise oxygen, carbon dioxide and one or more inert gases, such as nitrogen and argon. The measurement of the amount of inert gas, which we will refer to below as nitrogen (although in fact it may contain argon and other trace gases) may be obtained by measuring the amount of water vapour in the breath and estimating the amount of balance gas from the measured amounts of oxygen, carbon dioxide and water vapour.

The measurements of the amounts of respiratory gases may comprise measurements of the molar flows, or the total flow with concentrations or fractions of respiratory gases in breath. The measurements of the amounts of respiratory gases are preferably made at least every 50 ms, more preferably at least every 25 ms, more preferably at least every 10 ms.

The step of varying the parameters of the lung model to fit the predicted expired respiratory gas amounts to the plural measurements may comprise minimizing the differences, using a non-linear optimisation routine. For example a routine may be used which minimises the sum of the squares of the differences for one or more of the respiratory gases at each expiratory measurement time point. Preferably the step of varying the parameters of the lung model to fit the predicted expired respiratory gas amounts to the plural measurements comprises fitting the predicted expired respiratory gas amounts to the expiratory flow profile over each breath of the multiple successive breaths for one or more of the respiratory gases. In one embodiment the step of varying the parameters of the lung model to fit the predicted expired respiratory gas amounts to the plural measurements comprises fitting the predicted expired carbon dioxide and oxygen amounts measured during the period of steady breathing and fitting the inert gas amounts in the period of inert gas wash-in or wash-out.

A variety of wash-in and/or wash-out procedures may be used. For example, the period of steady breathing may be a period of, e.g., ten minutes, breathing air, this being followed by the period of wash-out by having the subject breathe pure oxygen (or air with added oxygen, e.g. 50%) for, e.g. five minutes, resulting in a wash-out of nitrogen. The model may be fitted to the oxygen and carbon dioxide measurements only during the steady breathing period and to the nitrogen measurements only during the wash-out period. Alternatively, there may be a period of breathing air mixed with a trace gas, such as methane or acetylene, which is also measured, e.g. for five minutes, resulting in a wash-in of the trace gas, followed by a period, e.g. of five minutes, of breathing air, resulting in a wash-out of the trace gas. In this case the model may be fitted to the oxygen, carbon dioxide and one or more trace gas measurements during these periods. Thus the period of steady breathing (i.e. stable oxygen and carbon dioxide) may be simultaneous with the period of inert gas wash-in and/or wash-out. In another approach, the supply of trace gas may be repeatedly added and withdrawn from the inspiratory gases such that wash-in and wash-out periods alternate.

Preferably the parameterized lung model models the lung as a plurality of alveolar compartments. More preferably these may be connected by a respective plurality of personal deadspaces to a common deadspace leading to the mouth. Preferably the volumes of the personal deadspaces may be distributed with alveolar compartment volume according to a personal deadspace distribution.

In the parameterized model the compartments are defined by one or more of: the volume of the common deadspace; the fractional volume of each alveolar compartment, being the fraction of the total alveolar volume of each compartment at the functional residual capacity of the model; the fractional expansion or compliance of each alveolar compartment, being approximately the fraction of the flow measured at the mouth received by each alveolar compartment; the fractional volume of the personal deadspace for each alveolar compartment, being the fraction of the total personal deadspace; the vascular conductance of each alveolar compartment, being the fraction of the total perfusion received by each compartment.

The model parameters may define: a bivariate distribution for the variation of fractional lung compliance with fractional volume and the variation of vascular conductance with fractional volume, the bivariate distribution being defined by the variance of the fractional lung compliance with fractional volume distribution, the variance of vascular conductance with fractional volume distribution, and their correlation. The bivariate distribution is preferably a lognormal distribution. Preferably in one embodiment the fractional compliance is used as the intrinsic property of the lung, and it is used to approximate the fractional distribution of ventilation.

In one embodiment the model parameters define a normal, or lognormal, distribution for the variation of the fractional personal deadspace volume with the fractional compartment volume.

While lognormal distributions are mentioned above and used in the illustrated embodiments below, other forms of distribution such as normal distributions, or functionally-defined distributions may be used to define the variation of fractional lung compliance with fractional volume, the variation of vascular conductance with fractional volume and the variation of the fractional personal deadspace volume with the fractional compartment volume.

To run the model preferably measurements of the inspired amounts and measured flow of respiratory gases are input to the parametrized model.

Another aspect of the invention provides an apparatus for quantifying lung impairment in accordance with the method of any one of the preceding claims and comprising:
a. a molecular flow sensor for obtaining measurements of the amounts at the mouth of plural respiratory gases in inhaled and exhaled breath as a function of time within each breath of a plurality of successive breaths during the period of steady breathing and the period of inert gas wash-in or wash-out of a respiring subject;
b. a data processor adapted to receive the concentration measurements and to model lung function using the parameterized lung model to predict respiratory gas amounts at the mouth of a respiring subject by varying the parameters of the lung model to fit the predicted expired respiratory gas amounts to the plural measurements; and adapted to output at least one parameter of the lung model as a quantifier of lung function.

Preferably the molecular flow sensor is adapted to measure the amounts of oxygen and carbon dioxide, and preferably also water vapour, in an airway at the mouth of a respiring subject and the data processor is adapted to calculate from the water vapour, oxygen and carbon dioxide measurements the amount of balance gas (principally nitrogen and argon).

The molecular flow sensor is preferably adapted to measure the molar flows, or the total flow with concentrations or fractions of respiratory gases in breath of a respiring subject. The molecular flow sensor may also be adapted to measure the amounts of other gases, which may be used as tracer gases, such as methane or acetylene.

In a preferred embodiment the molecular flow sensor is adapted to measure the amounts of respiratory gases at least every 50 ms, more preferably at least every 25 ms, more preferably at least every 10 ms.

The invention also extends to computer program comprising program code means, for example encoded on a tangible storage medium, for controlling a computer to execute the method of the invention by:
a. receiving measurements of the amounts at the mouth of plural respiratory gases in inhaled and exhaled breath as a function of time within each breath of a plurality of successive breaths during a period of steady breathing and a period of inert gas wash-in or wash-out of a respiring subject;
b. modelling lung function by using a parameterised lung model adapted to predict expired amounts of the plural respiratory gases at the mouth of a respiring subject at the time point of each of said plural measurements;
c. varying the parameters of the lung model to fit the predicted expired respiratory gas amounts to the plural measurements; and
d. outputting at least one parameter of the lung model as a quantifier of lung function.

Another aspect of the invention is the use of a quantified measure of lung inhomogeneity, preferably determined by the method of the invention, as a biomarker of lung disease. The quantified measure may comprise at least one of: a measure of the variation in lung compliance across the lung, a measure of the variation in deadspace fraction across the lung, a measure of the relative inefficiency of oxygen or carbon dioxide exchange between an inhomogeneous and homogeneous lung, and the difference in systemic arterial or mixed venous oxygen or carbon dioxide concentration between a homogenous and inhomogeneous lung.

The measure of the variation in lung compliance with volume may be the standard deviation or variance in the log of the distribution of lung compliance with alveolar volume and the measure of the variation in deadspace with volume may be the standard deviation or variance of the distribution of deadspace with alveolar volume.

Thus with the present invention inert gas wash-in or wash-out data are used to fit a mathematical model of the lung, producing parameter values that constitute quantitative biomarkers for lung function. In particular, while healthy lungs are relatively homogeneous structures, diseased lungs progressively lose homogeneity. Thus measures of inhomogeneity using the technique of the invention provide biomarkers for chronic airways disease.

The lung model is preferably assembled from a set of individual lung volumes which have associated with them: i) a set of fractional compliance values, which are preferably distributed log-normally; ii) a set of fractional vascular conductance values, which are preferably distributed log-normally; iii) a set of fractional deadspace values, which are preferably distributed normally. The compliance values and vascular conductance values are correlated with each other but the deadspace distribution is preferably not correlated with the compliance and vascular conductive distributions.

The outputs of interest from the invention, which constitute the biomarkers, are: the parameter values of the model together with values derived from them, preferably including: (i) the percent efficiency for gas exchange for carbon dioxide and oxygen compared with a homogenous lung; and (ii) the difference in systemic mixed venous or arterial partial pressures or contents for carbon dioxide and oxygen for the inhomogeneous lung compared with a homogenous lung. The calculation of (i) and (ii) is described below.
i) Assume the homogeneous lung has a single compartment with the total alveolar volume (at functional residual capacity, FRC) and total deadspace volume. Then compare the gas exchange of the homogeneous lung (under the assumption that the alveolar volume tracks that of the inhomogeneous lung and the venous concentrations remain the same).
ii) Find the systemic venous concentrations for the homogeneous lung that make the gas exchange for the homogeneous lung match that of the inhomogeneous lung and then compare the differences in venous/arterial concentrations or partial pressures.

In one embodiment, the invention preferably outputs the total volume of personal deadspace, the total alveolar volume, the standard deviation of the personal deadspace distribution, the standard deviation of the log normal compliance:volume distribution and the standard deviation of the log normal vascular conductance:volume distribution.

The present invention will be further described by way of example with reference to the accompanying drawings in which:—

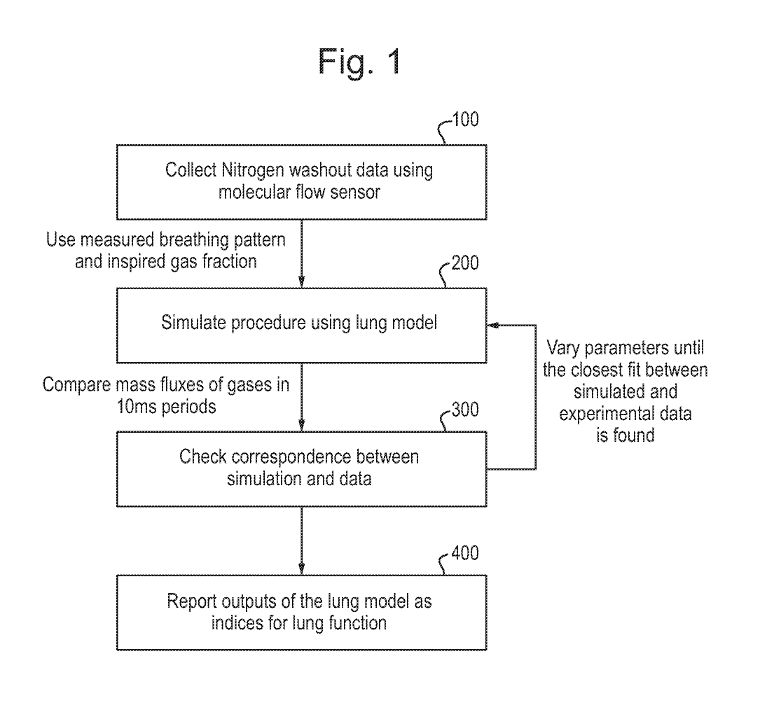

FIG. 1 is an outline of the process of one embodiment of the invention;

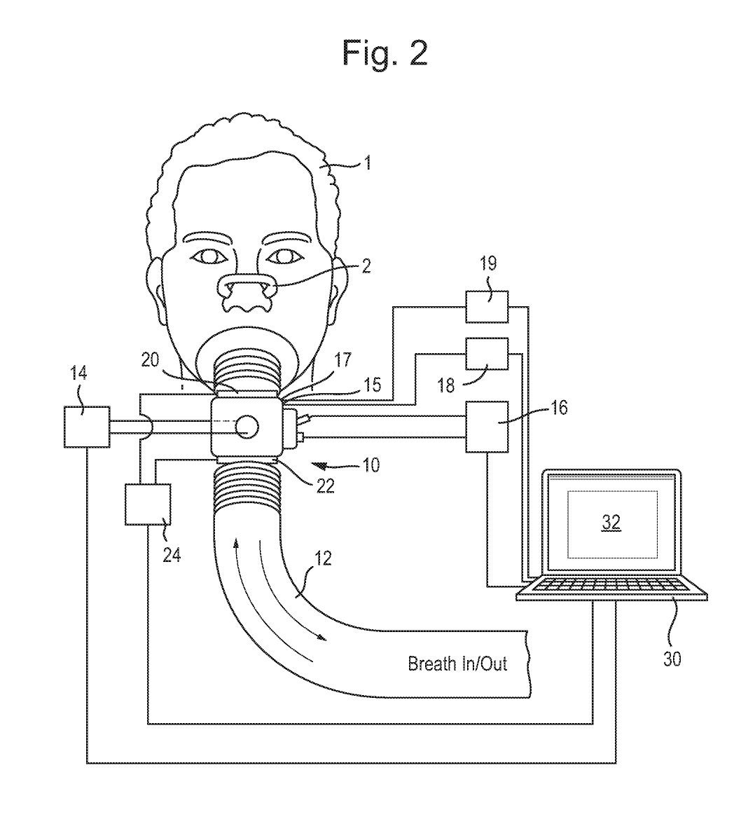

FIG. 2 schematically illustrates the apparatus of one embodiment of the invention in use;

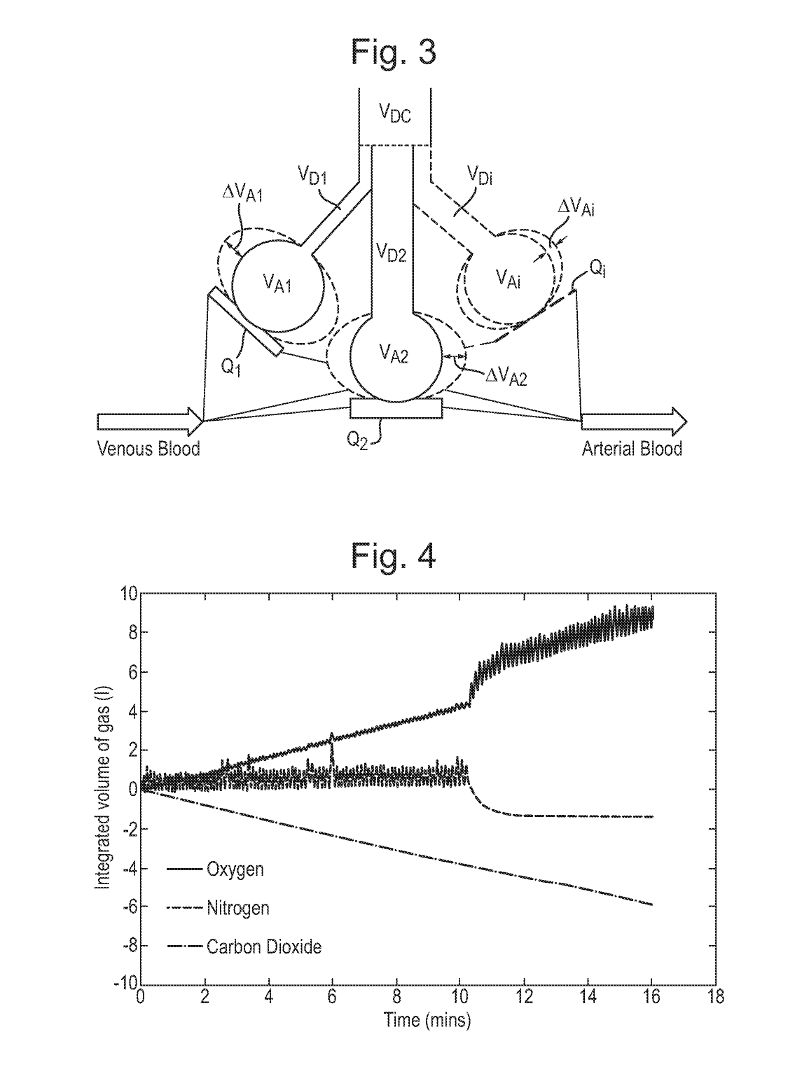

FIG. 3 schematically illustrates the basis of the lung model used in one embodiment of the invention;

FIG. 4 shows example measured wash-out data from a wash-out procedure in accordance with an embodiment of the invention;

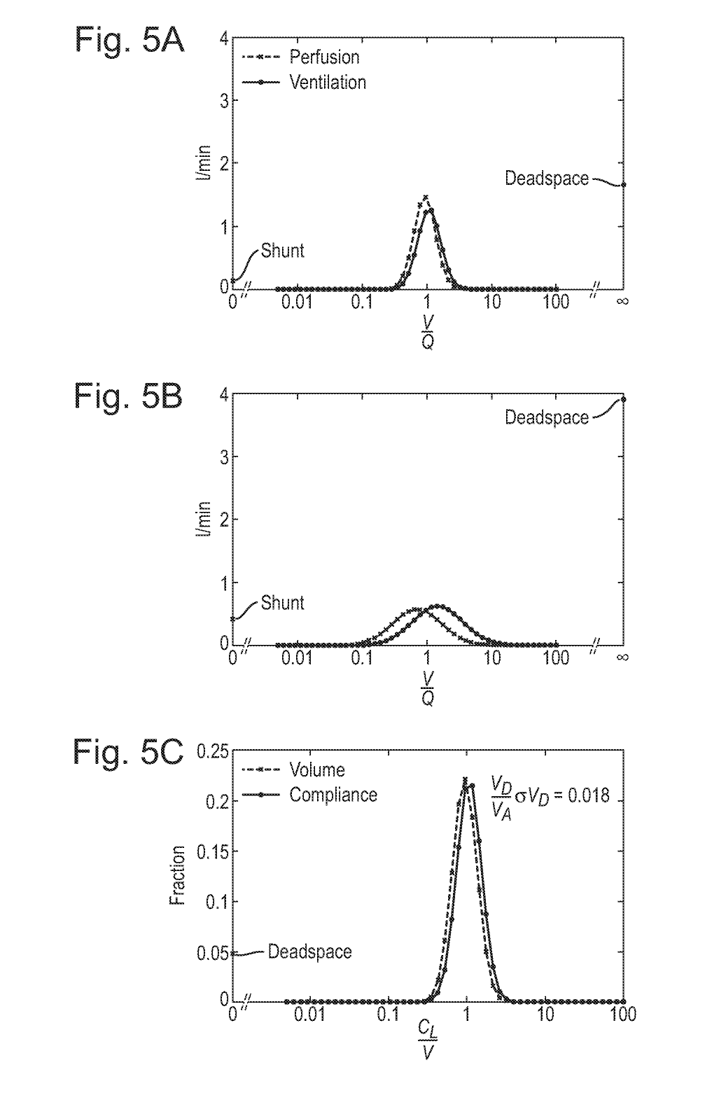

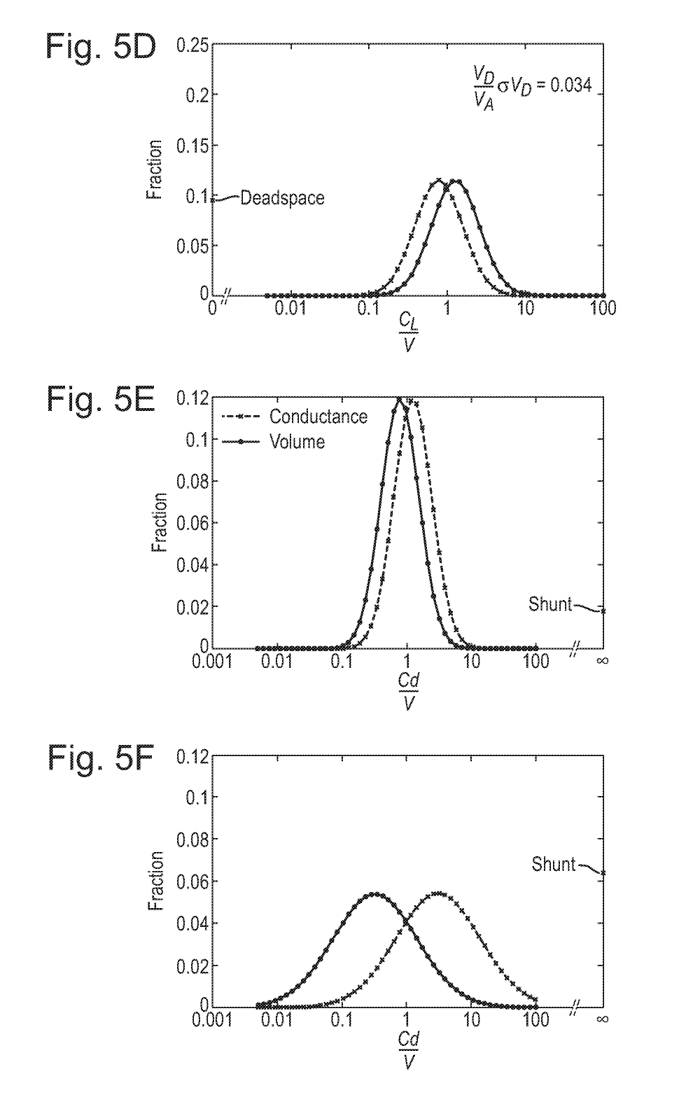

FIGS. 5A to F illustrate distributions for ventilation:perfusion, compliance:volume and vascular conductance:volume ratios for healthy (panels A, C, E) and COPD (panels B, D, F) subjects obtained by an embodiment of the invention;

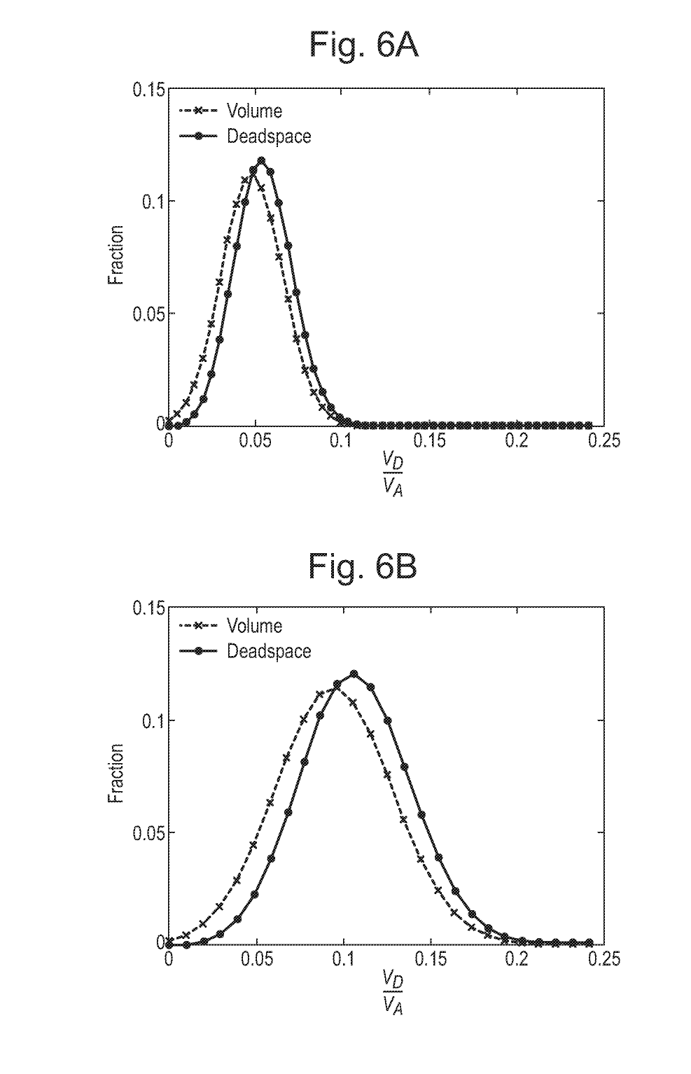

FIGS. 6A and B illustrate distributions for deadspace: volume ratios for healthy and COPD human subjects obtained by an embodiment of the invention;

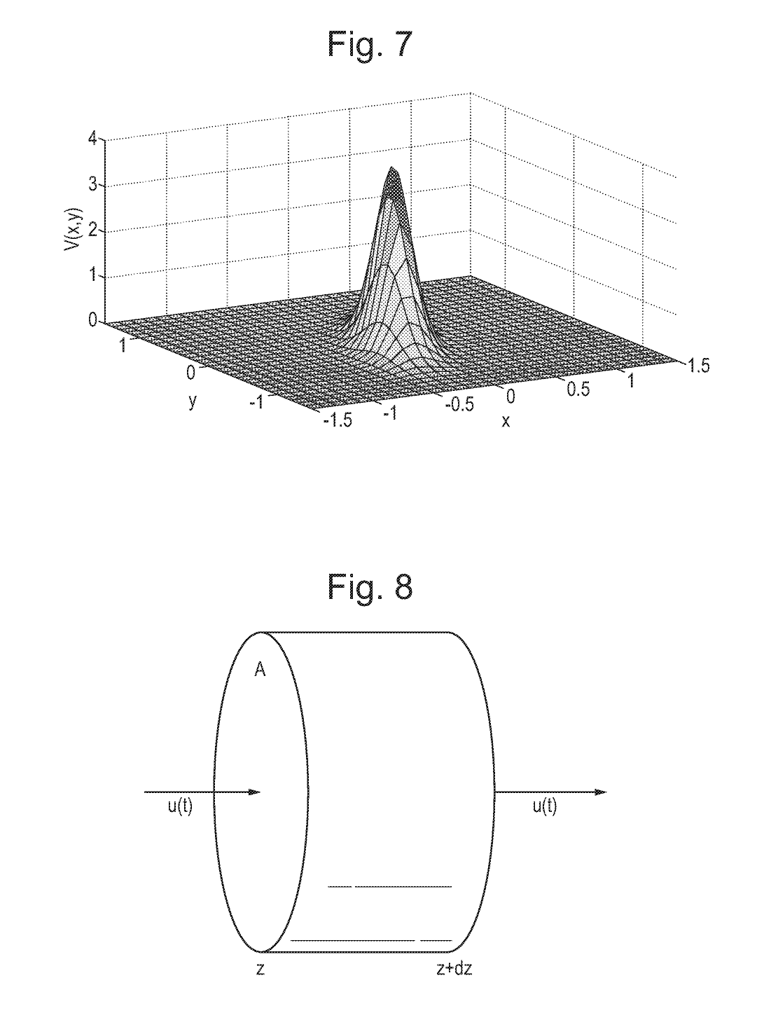

FIG. 7 illustrates a bivariate log-normal distribution; and

FIG. 8 illustrates a small region of a deadspace compartment in one embodiment of the invention.

This embodiment of the invention is based on the use of a pneumotachograph utilising a laser absorption spectrometer which can provide molecular flow sensing within the airway with an accuracy of better than 0.2% for flow and volume measurement and better than 0.5% for gas analysis, sampling every 10 ms. The high precision and high time resolution allow the molar flow of gas species in the airway to be monitored at the subject's mouth and these data are used to fit a mathematical model of the function of an inhomogeneous lung, which in turn provides accurate and new measures of lung inhomogeneity.

The process of one embodiment of the invention is outlined in FIG. 1. In step 100 inert gas wash-out data are collected using a highly accurate, high temporal resolution molecular flow sensor in the airway of a patient. The measured carbon dioxide, oxygen and inert gas flows, together with the subject's arterial oxygen saturation (measured using a pulse oximeter) are transferred to a data processor (such as a general purpose computer) running a simulation 200 of the function of an inhomogeneous lung. The data processor conducts an, e.g. non-linear, optimisation process 300 which finds the sum of the squares of the differences between the gas flows predicted by the simulation 200 and measured experimental values from the procedure 100 of the carbon dioxide and oxygen at each (10 ms) time point during an initial phase of air breathing, and the sum of the squares of the differences between the inert gas flows predicted by the simulation 200 and measured during a washout phase following the air breathing phase, and varies the parameters of the mathematical model used in the simulation 200 to minimise sum of the squares of the differences. The data processor reports, in process 400, the outputs and parameters of the mathematical model as indices of lung function.

FIG. 2 schematically illustrates the apparatus for the procedure 100. This embodiment utilises a molecular flow sensor of the type disclosed in Ciaffoni, L., O'Neill, D. P., Couper, J. H., Ritchie, G. A., Hancock, G., & Robbins, P. A. (2016), “In-airway molecular flow sensing: A new technology for continuous, non-invasive monitoring of oxygen consumption in critical care”, Science Advances, 2(8), e1600560. The main functional components of the molecular flow sensor are in a measurement head 10 positioned in an airway 12 of a subject 1. The measurement head 10 is connected to a controller 14 for controlling a laser absorption spectrometer (which is a cavity-enhanced absorption spectrometer) which is used in this embodiment for oxygen measurement, and a controller 16 is provided for controlling a light source and a light detector in a separate optical path used for carbon dioxide and water concentration measurements. On either side of the measurement head 10 are pressure sampling assemblies 20 and 22 connected to a differential pressure sensor 24. The measurement head 10 also includes a temperature sensor 15 for measuring the temperature of the gas flowing through the airway, and a barometric pressure sensor 17 for measuring the static pressure inside the measurement space, connected to respective controllers 18 and 19. The individual controllers 14, 16, 18, 19 and 24 are connected to a system controller 30 which controls the individual controllers, receives their outputs and calculates their results. The results may be displayed on display 32 and in this embodiment are output to a data processor. In particular the outputs are fluxes in litres (STPD) of oxygen, carbon dioxide, water vapour and balance (inert) gas every 10 ms.

Alternative embodiments may include sensors to measure other inert gases such as methane or acetylene which may be used as trace gases and in such cases the respiratory measurement procedure 100 of FIG. 1 alternatively uses such a trace gas as the inert gas in a wash-in followed by wash-out procedure, or alternating periods of wash-in and wash-out. In these cases the measurements of the inert gas may be simultaneous with the measurements of carbon dioxide and oxygen.

In practice a subject may need to be allowed to breathe steadily for 2 or 3 minutes to stabilize their breathing (some subjects hyperventilate initially in respiratory testing). Thus measurements during that period are not used. After the subject has settled-down a period of steady breathing may be used to set the model's initial conditions without varying the parameters of the lung model. Then the period of steady breathing followed by the period of inert gas wash-out may follow, or the periods of wash-in and wash-out of inert gases, with the gas flows being accurately measured for supply of the measurements to the data processor.

FIG. 4 illustrates example wash-out data output from the apparatus for a 16 minute period in which for the first 10 minutes the subject breathes constant-inspired oxygen and carbon dioxide (here, atmospheric air) and a subsequent 6 minute period of wash-out of an inert gas (mainly nitrogen) by breathing pure oxygen.

FIG. 3 illustrates the components of the inhomogeneous lung model used in this embodiment. To model heterogeneity within the lung, a lung of total alveolar volume VA is constructed from a set of N lung compartments of equal volume (at FRC) (only three lung compartments are illustrated but in a typical model there would be many—e.g. N=125), but which differ in their fractional shares of: total lung compliance (C_{L}) (compliance is the change in volume divided by the change in pressure), total pulmonary vascular conductance (C_{d}) (conductance is the blood flow per unit of driving pressure) and total deadspace (V_{D}). By using fractional quantities (i.e. the total is normalized to one) it is not necessary to look at absolute values for each compartment. The lung compartments are connected to a common deadspace V_{DC} and in turn to the molecular flow sensor, which has its own (known) deadspace.

To minimise the parameter count it is assumed based on previous studies (Wilson, JAP 72: 2298-2304, 1992; Beck JAP 90: 2151-2156, 2001; Beck JAP 113: 872-877, 2012), that both C_{L} and Cd have a continuous log-normal distribution at FRC. This results in a bivariate log-normal distribution for these parameters as illustrated in FIG. 7, where the correlation between the two distributions is defined as ρ. The standard deviations of C_{L} and Cd are σ_{CL/V}_{_{A}} and σ_{Cd/V}_{_{A}}, respectively. The variation of deadspace between compartments is described by a normal distribution with standard deviation σ_{V}_{_{D}}. We explain below how this continuous distribution is discretised into discrete lung compartments. The final core parameter of the lung required is the volume multiplier (Vtiss) for CO₂, which accounts for the additional effective lung volume that arises from the solubility of CO₂ within the lung tissue (Cander JAP 14: 541-551, 1959; Sackner JAP 19: 374-380, 1964). These parameters and their definitions are listed in Table 1.

TABLE 1

| Parameter | Definition |
|---|---|
| V_{A} = NV_{Ai} | Total alveolar volume when the lungs |
| | are at FRC, where N is the number |
| | of lung compartments. |
| ${V}_{tiss} = \frac{{V}_{A {Co}_{2}} - {V}_{A}}{{V}_{A}}$ | Volume multiplier for carbon dioxide to incorporate the effective lung tissue volume |
| ${Q}_{T} = {\sum }_{i = 1}^{N} {Q}_{i}$ | Cardiac output (Qᵢ is the blood flow associated with compartment i) |
| ${σ}_{{C}_{L} / {V}_{A}} = SD ( ln ( \frac{N Δ {V}_{Ai}}{\sum Δ {V}_{Ai}} ) )$ | Standard deviation of fractional compliance (C_{L}) distribution; where: |
| | ${C}_{L} = \sum {C}_{Li} = 1 and {C}_{Li} = \frac{Δ {V}_{Ai}}{\sum Δ {V}_{Ai}}$ |
| ${σ}_{{C}_{d} / {V}_{A}} = SD ( ln ( \frac{{NQ}_{i}}{\sum {Q}_{i}} ) )$ | Standard deviation of fractional vascular conductance C_{d} distribution; where: |
| | ${C}_{d} = \sum {C}_{di} = 1 and {C}_{di} = \frac{{Q}_{i}}{\sum {Q}_{i}}$ |
| $ρ = corr ( ln ( \frac{N Δ {V}_{Ai}}{\sum Δ {V}_{Ai}} ) , ln ( \frac{{NQ}_{i}}{\sum {Q}_{i}} ) )$ | Correlation of vascular conductance and compliance |
| V_{DC} | Apparatus deadspace volume |
| ${V}_{D} = {\sum }_{i = 1}^{N} {V}_{Di}$ | Total personal deadspace volume |
| ${σ}_{VD} = \frac{N}{{V}_{D}} SD ( {V}_{Di} )$ | Standard deviation of deadspace distribution |

To run the model, an alternating inspiratory and expiratory respiratory flow pattern and a pulmonary blood flow must be supplied. It also needs to be supplied with an inspiratory gas composition and a method for calculating pulmonary arterial blood gas composition (which will be explained below). The outputs of the model are the compositions of the expired gas during exhalation and the composition of pulmonary venous blood throughout the respiratory cycle. A fuller description of the model and the estimation of its parameters is given below.

### Detailed Structure of the Model and Description of Parameter Estimation Procedure

The fractional compliance (C_{Li}) and fractional pulmonary vascular conductance (C_{di}) of an alveolar compartment are determined by discretising a continuous, bivariate log normal distribution.

### Bivariate Lognormal Distribution

For ease of notation x and y are defined as:

$$x = ln ( \frac{{C}_{L} ( x )}{V ( x , y )} ) \\ y = ln ( \frac{{C}_{d} ( y )}{V ( x , y )} ) , \qquad ( E1 )$$

where C_{L}(x) is the fractional compliance, C_{d}(y) is the fractional pulmonary vascular conductance, and V(x,y) is the fractional alveolar volume at FRC.

The governing distribution of the model V(x,y) is defined with the statement that V(x,y)dxdy is the fraction of the alveolar volume with

$$x = log ( \frac{{C}_{L}}{V} ) and y = log ( \frac{{C}_{d}}{V} )$$

values that lie in small intervals of length dx and dy around the values of x and y.

It is assumed that V is a bivariate lognormal distribution of

$$\frac{{C}_{L}}{V} and \frac{{C}_{d}}{V} ,$$

thus:

$$V ( x , y ) = \frac{1}{2 {πσ}_{{C}_{L} / {V}_{A} {σ C}_{d} / {V}_{A}} \sqrt{1 - {p}^{2}}} exp ( \frac{- 1}{2 ( 1 - {ρ}^{2} )} ( \frac{{( x - \overline{x} )}^{2}}{{σ}_{{C}_{L} / {V}_{A}}^{2}} - \frac{2 ρ ( x - \overline{x} ) ( y - \overline{y} )}{{σ}_{{C}_{L} / {V}_{A}} {σ}_{{C}_{d} / {V}_{A}}} + \frac{{( y - \overline{y} )}^{2}}{{σ}_{{C}_{d} / {V}_{A}}^{2}} ) ) , \qquad ( E2 )$$

where σ_{C}_{_{L}}_{/V}_{_{A}}² and σ_{C}_{_{d}}_{/V}_{_{A}}² are the variances of the compliance-volume and vascular conductance-volume distributions respectively, x̅ and y̅ are their means, and ρ the coefficient of correlation between x and y.

The compliance-volume (V(x)) and vascular conductance-volume (V(y)) distributions are the projections of V(x,y) onto the x and y axes respectively. Thus,

V(x)=∫_{-∞}^{+∞}V(x,y)dy, (E3)

and

V(y)=∫_{-∞}^{+∞}V(x,y)dx, (E4)

Substituting (E2) into these expressions we have

$$\begin{matrix}V ( x ) = \frac{1}{\sqrt{2 π} {σ}_{{C}_{L} / {V}_{A}}} exp ( \frac{{( x - \overline{x} )}^{2}}{{σ}_{{C}_{L} / {V}_{A}}^{2}} ) , & ( E5 ) \\ and & \\ V ( y ) = \frac{1}{\sqrt{2 π} {σ}_{{C}_{d} / {V}_{A}}} exp ( \frac{{( y - \overline{y} )}^{2}}{{σ}_{{C}_{d} / {V}_{A}}^{2}} ) , & ( E6 )\end{matrix}$$

Thus V(x) and V(y) are univariate lognormal distributions.

As will now be shown, this distribution is described by only three independent parameters. From (E1),

C_{L}(x)=V(x)e^{x}, (E7)

substituting (E5) into this expression and rearranging:

$${C}_{L} ( x ) = \frac{1}{\sqrt{2 π} {σ}_{{C}_{L} / {V}_{A}}} exp ( \frac{- {( x - ( \overline{x} + {σ}_{{C}_{L} / {V}_{A}}^{2} ) )}^{2}}{2 {σ}_{{C}_{L} / {V}_{A}}} ) exp ( \frac{( ( {σ}_{{C}_{L} {V}_{A}}^{2} - 2 \overline{x} ) )}{2} ) . \qquad ( E8 )$$

Since it is required that

$$\begin{matrix}{{\int }_{- ∞}}^{+ ∞} {C}_{L} ( x ) dx = 1 , & ( E9 ) \\ then & \\ exp ( \frac{{σ}_{{C}_{L} / {V}_{A}}^{2} + 2 \overline{x}}{2} ) = 1 , & ( E10 ) \\ and so & \\ \overline{x} = - \frac{{σ}_{{C}_{L} {V}_{A}}^{2}}{2} . & ( E11 )\end{matrix}$$

Since from (E1),

C_{d}(y)=V(y)e^{y}, (E12)

an analogous procedure gives:

$$\overline{y} = - \frac{{σ}_{{C}_{d} / {V}_{A}}^{2}}{2} . \qquad ( E13 )$$

In contrast to previous work the means of the distribution, x̅ and y̅, are not independent parameters, and the distribution is governed by three parameters: σ_{C}_{_{L}}_{/V}_{_{A}} and ρ. This is because fractional quantities are being used, and the distribution is assumed as independent of the ventilation and the cardiac output.

The distribution (E2) must be discretized to provide the compliances and conductances for each of the compartments of the model with NₓN_{y} unique pairs of compliance-volume and vascular conductance-volume ratios. Each of these pairs is indexed with the subscripts ij, where i=1, 2, . . . Nₓ and j=1, 2, . . . N_{y}. Various methods may be used to discretize the distribution, but two methods are described below.

### A Straightforward Discretisation

First x and y are discretised into Nₓ+1 and N_{y}+1 equally spaced points. Since these points are equally spaced finite upper and lower bounds must be chosen, and thus x₀ is set such that

∫_{-∞}^{x}^{₀}V(x)dx=0.0001, (E14)

and x_{N}_{ₓ} such that

$${{\int }_{{x}_{{N}_{x}}}}^{∞} V ( x ) dx = 0.0001 . \qquad ( E15 )$$

The values y₀ and y_{N}_{_{y}} are defined by analogous expressions involving the relevant univariate distribution V(y).

The fractional volume of compartment (ij) when the lung is at FRC can then be defined as,

V_{i,j}=∫_{y}_{_{j−1}}^{y}^{ⱼ}∫ₓ_{_{i−1}}^{x}^{ᵢ}V(x,y)dxdy, (E16)

Using the definitions of x and y given by (E1), the compliance (C_{L}_{_{i,j}}) is

C_{L}_{_{i,j}}=∫_{y}_{_{j−1}}^{y}^{ⱼ}∫ₓ_{_{i−1}}^{x}^{ᵢ} exp(x)V(x,y)dxdy, (E17)

and the vascular conductance C_{d}_{_{i,j}},

C_{d}_{_{i,j}}=∫_{y}_{_{j−1}}^{y}^{ⱼ}∫ₓ_{_{i−1}}^{x}^{ᵢ} exp(y)V(x,y)dxdy. (E18)

This method of discretising the continuous distribution works particularly well if the major and minor axes of the elliptical probability density function lie along the x and y axes (i.e. if ρ=0). However if ρ≠0 the probability density function is an ellipse whose major and minor axes do not lie along the x and y axes. In this case the approach described above, in which the distribution is discretized into equal-sized rectangles along the x and y axes, will be quite inefficient since there will be a number of compartments with almost zero volume, and one or two containing the majority of the volume.

Thus in the next section advantage is taken of a geometrical property of the normal distribution that allows a change of variables so that the distribution can be discretised more efficiently.

### Discretisation by Change of Variables

To discretise the governing continuous distribution more efficiently, advantage is taken of a useful property of the bivariate normal distribution. This property is that any bivariate normal distribution can be represented as a transformation of the standard bivariate normal distribution (zero means, zero covariance, and unit variances). In this section the outline given in “The multivariate Gaussian distribution” by C. B. Do is followed, and we explain how this change of variables may be made by formulating the bivariate normal distribution in vector notation. The integrals that must be solved to define the compartments in these new variables are then set-up.

The bivariate distribution, (E2), can be written in the following notation:

$$\begin{matrix}V ( x ) = \frac{1}{2 π {\vert \sum \vert}^{\frac{1}{2}}} exp ( - \frac{1}{2} {( x - μ )}^{T} {\sum }^{- 1} ( x - μ ) ) , & ( E19 ) \\ where & \\ x = ( \begin{matrix}x \\ y\end{matrix} ) . & ( E20 ) \\ μ = ( \begin{matrix}\overline{x} \\ \overline{y}\end{matrix} ) & ( E21 ) \\ \sum = ( \begin{matrix}{σ}_{{C}_{L} : {V}_{A}}^{2} & {ρσ}_{{C}_{L} : {V}_{A}} {σ}_{{C}_{d} : {V}_{A}} \\ {ρσ}_{{C}_{L} : {V}_{A}} {σ}_{{C}_{d} : {V}_{A}} & {σ}_{{C}_{d} : {V}_{A}}^{2}\end{matrix} ) . & ( E22 )\end{matrix}$$

The contour lines of constant probability are ellipses centred on the mean, with major and minor axes defined by the eigenvectors of Σ.

To perform a change of variables we first diagonalise Σ,

Σ=UΛU^{T}, (E23)

where Λ is a diagonal matrix with, as 0≤ρ≤1, positive entries on the diagonal and U is a rotation matrix, and so

UU^{T}=I, (E24)

where I is the identity matrix. We can re-write (E23) as

$$\begin{matrix}\sum = ( U {Λ}^{\frac{1}{2}} ) {( U {Λ}^{\frac{1}{2}} )}^{T} , & ( E25 ) \\ where & \\ {Λ}^{\frac{1}{2}} = ( \begin{matrix}\sqrt{{Λ}_{11}} & 0 \\ 0 & \sqrt{{Λ}_{22}}\end{matrix} ) . & ( E26 )\end{matrix}$$

If we substitute (E25) into (E19) and use the fact that the determinant of a matrix's transpose is equal to the determinant of the matrix, we have

$$V ( x ) = \frac{1}{2 π \vert ( U {Λ}^{\frac{1}{2}} ) \vert} exp ( - \frac{1}{2} {( x - μ )}^{T} {( U {Λ}^{\frac{1}{2}} )}^{- T} {( U {Λ}^{\frac{1}{2}} )}^{- 1} ( x - μ ) ) . \qquad ( E27 )$$

To simplify this expression we define a new set of axes s,

$$\begin{matrix}s = {( U {Λ}^{\frac{1}{2}} )}^{- 1} ( x - μ ) , & ( E28 ) \\ where & \\ s = ( \begin{matrix}s \\ t\end{matrix} ) , & ( E29 )\end{matrix}$$

and thus multiplying (E28) by UΛ^{1/2} we have

x=μ+UΛ^{1/2}s, (E30)

The Jacobian of this transformation is J, where

$$J = ( \begin{matrix}\frac{\partial x}{\partial s} & \frac{\partial x}{\partial t} \\ \frac{\partial y}{\partial s} & \frac{\partial y}{\partial t}\end{matrix} ) . \qquad ( E31 )$$

Calculating this explicitly from (E30) we have that,

J=UΛ^{1/2}. (E32)

To change the variables of a probability density function (PDF), the PDF in the old variables is multiplied by the determinant of the Jacobian for the transformation between the two sets of variables. Thus in our case,

V(s)=V(x)|J|. (E33)

Using this and substituting for s in (E27), we obtain

$$V ( s ) = \frac{1}{2 π} exp ( - \frac{1}{2} {s}^{T} s ) . \qquad ( E34 )$$

This is the standard bivariate normal distribution (zero means, zero covariance, and unit variances) defined on s. Thus any bivariate normal distribution defined on x can be represented as the standard bivariate normal distribution on s, using a suitable change of variables. The advantage of using the standard bivariate normal distribution is that the major axes of the ellipses that form the probability density function are along the axes on which we define the distribution.

We can then define a new rectangular grid on s and t, with Nₛ intervals in the s direction and Nₜ intervals in the t direction, which samples the distribution effectively, and replace the integrals discussed above to find the compliances, volumes, and vascular conductances associated with these (NₛNₜ) rectangles (compartments).

In the new variable s and t we can replace (E16) with

Vᵢⱼ=∫ₜ_{_{j−1}}^{t}^{_{j∫}}ₛ_{_{i−1}}^{s}^{ᵢ}V(s,t)dsdt, (E35)

where i=1 . . . Nₛ, j=1 . . . Nₜ, and

$$V ( s , t ) = \frac{1}{2 π} exp ( \frac{- {s}^{2} - {t}^{2}}{2} ) . \qquad ( E36 )$$

We can factorise (E35) into the product of two univariate integrals. If we do this we have

$${V}_{ij} = {{\int }_{{t}_{j - 1}}}^{{t}_{j}} \frac{1}{\sqrt{2 π}} exp ( \frac{- {t}^{2}}{2} ) dt {{\int }_{{s}_{i - 1}}}^{{s}_{i}} \frac{1}{\sqrt{2 π}} exp ( \frac{- {s}^{2}}{2} ) ds . \qquad ( E37 )$$

In the new variables we can then define the limits on the integrals such that each compartment represents an equal fraction of the alveolar volume at FRC

$$( i . e . {V}_{ij} = \frac{1}{{N}_{s} {N}_{t}} ) ,$$

rather than the compartments defined in the first discretization scheme above which represented equally sized rectangles on x and y. To do this we define sᵢ such that

$${\int }_{{s}_{i - 1}}^{{s}_{i}} \frac{1}{\sqrt{2 π}} exp ( \frac{- {s}^{2}}{2} ) ds = \frac{1}{{N}_{s}} . \qquad ( E38 )$$

In the new variables the limits are no longer equally spaced, and so s₀=−∞ and s_{N}_{ₛ}=∞. Similarly we define tⱼ such that

$${\int }_{{t}_{j - 1}}^{{t}_{j}} \frac{1}{\sqrt{2 π}} exp ( \frac{- {t}^{2}}{2} ) dt = \frac{1}{{N}_{t}} , \qquad ( E39 )$$

where t₀=−∞ and t_{N}_{ₜ}=∞.

Changing the variables of (E7) using (E30) and (E32) we have,

C_{L}(s,t)=exp(x̅+J₁₁s+J₁₂t)V(s,t) (E40)

Thus integrating between the limits on s and t we can discretise this expression to define the fractional compliance of compartment ij (C_{Lij}) as

C_{Lij}=∫ₜ_{_{j−1}}^{t}^{ⱼ}∫ₛ_{_{i−1}}^{s}^{ᵢ} exp(x̅+J₁₁s+J₁₂t)V(s,t)dsdt. (E41)

Similarly changing the variables of (E12) using (E30) and (E32), and integrating the fractional vascular conductance of compartment ij (C_{dij}) is

C_{dij}=∫ₜ_{_{j−1}}^{t}^{ⱼ}∫ₛ_{_{i−1}}^{s}^{ᵢ} exp(y̅+J₂₁s+J₂₂t)V(s,t)dsdt. (E41)

### Discretisation of the Deadspace

The total volume of personal deadspace associated with each of the N_{S}Nₜ unique pairs of compliance-volume and vascular conductance-volume ratios is VᵢⱼV_{D}. However, alveolar compartments with the same compliance-volume and vascular conductance-volume ratios may be located in different regions of the lung. Thus they will be associated with different volumes of deadspace. In this embodiment it is assumed that rather than forming a single compartment, this deadspace is also distributed, according to a personal deadspace distribution.

This distribution is discretised into N_{d} compartments for each pair of compliance-volume and vascular conductance-volume ratios; thus the total number of both alveolar and deadspace compartments, N, is NₛNₜN_{d}. The deadspace distribution is assumed to be a normal distribution and identical for each pair of compliance-volume and vascular conductance-volume ratios.

Defining:

$$z = \frac{{v}_{ij}}{{V}_{ij}} , \qquad ( E43 )$$

where vᵢⱼ is the fractional deadspace volume of an alveolar compartment

$$( \frac{{V}_{Di}}{{V}_{D}} ) ,$$

and thus

vᵢⱼ=zVᵢⱼ, (E44)

Assuming the fractional volume is normally distributed,

$${V}_{ij} ( z ) = \frac{{V}_{ij}}{2 {πσ}_{{V}_{D}} } exp ( \frac{- {( z - \overline{{z}_{ι J}} )}^{2}}{2 {σ}_{{V}_{D}}^{2}} ) , \qquad ( E45 )$$

Substituting (E43) into this expression:

$${v}_{ij} ( z ) = \frac{{V}_{{ij}^{z}}}{\sqrt{2 π} {σ}_{{V}_{D}} } exp ( \frac{- {( z - \overline{{z}_{ι J}} )}^{2}}{2 {σ}_{{V}_{D}}^{2}} ) . \qquad ( E46 )$$

The total fractional deadspace volume associated with each unique pair of C_{Lij}/Vᵢⱼ and C_{dij}/Vᵢⱼ ratios is Vᵢⱼ. Thus:

∫_{-∞}^{∞}vᵢⱼ(z)dz=Vᵢⱼ. (E47)

Letting=z−z̅ᵢ̅ⱼ̅:

$$\begin{matrix}{\int }_{- ∞}^{∞} \frac{{V}_{ij} ( Z + \overline{{z}_{ι J}} )}{\sqrt{2 π} {σ}_{{V}_{D}}} exp ( \frac{- {Z}^{2}}{2 {σ}_{{V}_{D}}^{2}} ) d Z = {V}_{ij} , \\ hence & ( E48 ) \\ {V}_{ij} = \overline{{z}_{ι J}} {V}_{ij} + {\int }_{- ∞}^{∞} \frac{{V}_{ij} Z}{\sqrt{2 π} {σ}_{{V}_{D}}} exp ( \frac{- {Z}^{2}}{2 {σ}_{{V}_{D}}^{2}} ) d Z , & ( E49 )\end{matrix}$$

and since the integrand is an odd function:

z̅ᵢⱼ=1. (E50)

Thus the normal deadspace distribution introduces one additional parameter, σ_{V}_{_{D}}.

The continuous deadspace distribution, (E46), is now discretized into discrete compartments. As each of the NₛNₜ compartments (denoted with indices ij) is split into N_{d}compartments, a third index k is introduced. Again assuming that each alveolar compartment has equal volume, the fractional volume of compartment ijk, Vᵢⱼₖ, is

$${V}_{ijk} = {\int }_{{z}_{k - i}}^{{z}_{k}} {V}_{ij} ( z ) dz = \frac{{V}_{ij}}{{N}_{d}} , \qquad ( E51 )$$

with z₀=−∞ and z_{N}_{_{d}}=∞.

The fractional deadspace volume (vᵢⱼₖ) associated with compartment ijk can then be defined as

vᵢⱼₖ=∫_{z}_{_{k−t}}^{z}^{ₖ}vᵢⱼ(z)dz. (E52)

The vascular conductance-volume and compliance-volume ratios of the NₛNₜ compartments (denoted with indices ij) do not change when each of them is split into N_{d} compartments (as these are set by the governing alveolar distribution), and thus we have

$${C}_{dijk} = {C}_{dij} \frac{{V}_{ijk}}{{V}_{ij}} \qquad ( E53 )$$

Similarly the fractional compliance is

$${C}_{Lijk} = {C}_{Lij} \frac{{V}_{ijk}}{{V}_{ij}} \qquad ( E54 )$$

The normal deadspace distribution (E45) is defined from −∞<z<∞. From (E43) we see that z<0 gives negative values of

$$\frac{v}{V} .$$

Thus if the value of σ_{V}_{_{D}} is sufficiently large (the larger the value of σ_{V}_{_{D}} the greater the density of the distribution away from its mean of one), then (E52) may give a deadspace compartment with a negative volume, which is clearly not physically realistic. Thus a maximum value for σ_{V}_{_{D}} is set that prevents (E52) giving a compartment with a negative volume.

### Note on Indexing of Compartments

This section began referring to compartments with two indices, ij, due to the discretisation of the bivariate lognormal distribution. A third index, k, was introduced as each one of these ij compartments was split into N_{d} compartments due to the discretisation of the deadspace distribution. Thus each compartment had three indices, ijk, and the total number of compartments is Nₛ×Nₜ×N_{d}.

This indexing was convenient for explaining the discretisation of the distributions. However, for simplicity of notation, in the rest of the document each compartment is uniquely identified with a single index î, where

î=i+Nₛ(j−1)+NₛNₜ(k−1), (E55)

where
i=1, 2, . . . , Nₛ,
j=1, 2, . . . , Nₜ,
k=1, 2, . . . , N_{d}.

In the rest of the document the \hat{ } is dropped and i (given by (E55)) will be the sole index used to refer to the compartments.

### Governing Equations

In this section the governing equation of the alveolar compartments for this embodiment will first be described, followed by how the fractional ventilation (the fraction of the flow measured at the mouth entering/leaving an alveolar compartment) is calculated from the compartment's compliance. The governing equations of the body's gas stores and deadspace compartments used in this embodiment are then given.

### Alveolar Compartments

As indicated above, it is assumed that the alveolar compartments are perfectly mixed, and that the gas exchange between the blood and the alveolar compartment reaches equilibrium. Since N₂ and O₂ are almost insoluble in lung tissue it is assumed that the volume of these gases dissolved in lung tissue will be negligible. A similar approach can be taken for any other relatively insoluble tracer gases that may be present. By conservation of mass, the rate of change of the volume of a given gas in a lung compartment is equal to the sum of the flux of that gas entering via ventilation from the personal deadspace, and the net flux of that gas due to exchange with the blood across the alveolar membrane. Thus for N₂ and O₂ during inspiration:

$$\frac{d ( {F}_{i}^{g} ( t ) {V}_{Ai} ( t ) )}{dt} = {\dot{V}}_{t}^{in} u ( t ) {F}_{Ii}^{g} ( t ) + {Q}_{T} {C}_{di} ( {C}_{\overline{v}}^{g} ( t ) - {C}_{ai}^{g} ( t ) ) , \qquad ( E56 )$$

where Fᵢ^{g}(t) is the gas fraction g (where g may be O₂ or N₂) in compartment i at time t, \dot{V}ₜⁱⁿ is the fraction of the flow measured at the mouth that enters compartment i during inspiration (the calculation of this quantity is discussed below), F_{Ii}^{g}(t) is the fraction of gas g inspired into compartment i from the connected personal deadspace compartment, Q_{T} is the cardiac output, C_{v̅}^{g}(t) is the mixed venous (pulmonary arterial) gas concentration of gas g entering the compartments, Cₐᵢ^{g}(t) the blood gas concentration of gas g leaving compartment i, V_{Ail (t) the volume of compartment i, and u(t) is the flow rate of gas (measured by the in-airway molecular flow sensing device (MFS)). It should be noted that since gas exchange with the blood is considered,}

$$\frac{{dV}_{Ai} ( t )}{dt}$$

is not equal to \dot{V}ₜⁱⁿu(t), as is often assumed, but is given by (E35), which is derived below.

However, CO₂ is significantly more soluble than N₂ or O₂, and thus is present in appreciable volumes in lung tissue. It is assumed that the tissue volume is a constant fraction (b) of the total alveolar volume at FRC (V_{A}) and distributed among the compartments in proportion to their volume at FRC. It is assumed that the partial pressures of CO₂ in the tissue and the alveoli are at equilibrium, and thus the volume of CO₂ dissolved in the tissue associated with compartment i is

VᵢV_{A}bλₜ(P_{b}−P_{H2O})Fᵢ^{CO}^{₂}(t), (E57)

where λₜ is the solubility of CO₂ in lung tissue. The volume multiplier for CO₂ to reflect the effective lung tissue volume within which CO₂ is distributed, Vₜᵢₛₛ, is then defined as

Vₜᵢₛₛ=bλₜ(P_{b}−P_{H2O}). (E58)

An analogous approach may be adopted for any soluble tracer gases that may be present.

Therefore, including this volume, for CO₂ on inspiration, by analogy with (E56):

$$\frac{d ( {F}_{i}^{{CO}_{2}} ( t ) ( {V}_{Ai} ( t ) + {V}_{i} {V}_{tiss} {V}_{A} ) )}{dt} = {\dot{V}}_{i}^{in} u ( t ) {F}_{Ii}^{{CO}_{2}} ( t ) + {Q}_{T} {C}_{di} ( {C}_{\overline{v}}^{{CO}_{2}} ( t ) - {C}_{ai}^{{CO}_{2}} ( t ) ) , \qquad ( E59 )$$

Summing (E56) (for both N₂ and O₂) and (E59), re-arranging, and remembering that F^{CO2}+F^{N2}+F^{O2}=1, gives:

$$\frac{{dV}_{Ai} ( t )}{dt} = {\dot{V}}_{i}^{in} u ( t ) + {Q}_{T} {C}_{di} {\sum }_{g} ( {C}_{\overline{v}}^{g} ( t ) - {C}_{ai}^{g} ( t ) ) - {V}_{i} {V}_{tiss} {V}_{A} \frac{{dF}_{i}^{{CO}_{2}} ( t )}{dt} , \qquad ( E60 )$$

where g denotes one of the three respiratory gases. Thus as noted earlier

$$\frac{{dV}_{Ai} ( t )}{dt}$$

is not equal to \dot{V}ᵢⁱⁿu(t).

On expiration the flux due to ventilation is leaving rather than entering the compartment. Thus (E56) becomes:

$$\frac{d ( {F}_{i}^{g} ( t ) {V}_{Ai} ( t ) )}{dt} = {\dot{V}}_{i}^{ex} u ( t ) {F}_{i}^{g} ( t ) + {Q}_{T} {C}_{di} ( {C}_{\overline{v}}^{g} ( t ) - {C}_{ai}^{g} ( t ) ) , \qquad ( E61 )$$

where \dot{V}ᵢ^{ex} is the fraction of the flow measured at the mouth that leaves compartment i during expiration (the calculation of this quantity is discussed below), and g is O₂ or N₂. Similarly for CO₂:

$$\frac{d ( {F}_{i}^{{CO}_{2}} ( t ) ( {V}_{Ai} ( t ) + {V}_{i} {V}_{tiss} {V}_{A} ) )}{dt} = {\dot{V}}_{i}^{ex} u ( t ) {F}_{i}^{{CO}_{2}} ( t ) + {Q}_{T} {C}_{di} ( {C}_{\overline{v}}^{{CO}_{2}} ( t ) - {C}_{ai}^{{CO}_{2}} ( t ) ) . \qquad ( E62 )$$

Summing (E61) (for both N₂ and O₂) and (E62), and re-arranging, gives the governing equation for compartmental volumes on expiration:

$$\frac{{dV}_{Ai} ( t )}{dt} = {\dot{V}}_{i}^{ex} u ( t ) + {Q}_{T} {C}_{di} {\sum }_{g} ( {C}_{\overline{v}}^{g} ( t ) - {C}_{ai}^{g} ( t ) ) - {V}_{i} {V}_{tiss} {V}_{A} \frac{{dF}_{i}^{{CO}_{2}} ( t )}{dt} . \qquad ( E63 )$$

### Fractional Ventilation

We assume constant values for \dot{V}ᵢ (the fraction of the flow measured at the mouth entering/leaving an alveolar compartment) during both expiration and inspiration, although the values for expiration and inspiration will be different, and are updated on a breath by breath basis as explained below.

The values for \dot{V}ᵢ are initialized by setting them equal to the values of C_{Li}, since the volume change during a single breath due to ventilation is much larger than that due to net gas exchange with the blood. On subsequent breaths, the value of \dot{V}ᵢ during inspiration (\dot{V}ᵢⁱⁿ) is the value that would have led the following integral to hold on the previous breath:

$${\int }_{0}^{{t}_{I}} \frac{{dV}_{Ai} ( t )}{dt} dt = {\int }_{0}^{{t}_{I}} {C}_{Li} \frac{{dV}_{Ai} ( t )}{dt} dt , \qquad ( E64 )$$

where t_{I} is the duration of that inspiration. Substituting in (E60), integrating, and re-arranging we have:

$${\dot{V}}_{i}^{in} = \frac{\begin{matrix}{C}_{Li} ( {V}_{A} ( {t}_{I} ) - {V}_{A} ( 0 ) ) - {\int }_{0}^{{t}_{I}} {Q}_{T} {C}_{di} {\sum }_{g} ( {C}_{\overline{v}}^{g} ( t ) - {C}_{ai}^{g} ( t ) ) + \\ {V}_{i} {V}_{tiss} {V}_{A} \frac{{dF}_{i}^{{CO}_{2}} ( t )}{dt} dt\end{matrix}}{{\int }_{0}^{{t}_{I}} u ( t ) dt} \qquad ( E65 )$$

Analogously on expiration:

$${\dot{V}}_{i}^{ex} = \frac{\begin{matrix}{C}_{Li} ( {V}_{A} ( {t}_{b} ) - {V}_{A} ( {t}_{I} ) ) - {\int }_{{t}_{I}}^{{t}_{b}} {Q}_{T} {C}_{di} {\sum }_{g} ( {C}_{\overline{v}}^{g} ( t ) - {C}_{ai}^{g} ( t ) ) + \\ {V}_{i} {V}_{tiss} {V}_{A} \frac{{dF}_{i}^{{CO}_{2}} ( t )}{dt} dt\end{matrix}}{{\int }_{{t}_{I}}^{{t}_{b}} u ( t ) dt} . \qquad ( E66 )$$

The values of these expression are estimated at the end of each breath and the calculated values of \dot{V}rᵢⁱⁿ and \dot{V}ᵢ^{ex} used on the subsequent inspiration/expiration.

### The Body's Gas Stores

Above are derived the governing equations for the alveolar compartments, and it can be seen that they depend on the venous gas concentrations C_{v̅}^{g}(t). During the air breathing phase of the washout procedure 100, these values are relatively stable and can be calculated from the data without the need for a model of recirculation. In the case of a nitrogen washout procedure using a high content of, or pure, oxygen as the inspiratory gas, the concentrations of both N₂ and O₂ in the pulmonary venous blood change substantially, and so models for recirculation must be employed.

To model the body's inert gas stores, in this embodiment the nitrogen stores, and their exchange with the blood, a known model (and parameters) such as that of e.g. Baker and Farmery, Comprehensive Physiology, 2011 is used. This model assumes that the body is made up of four compartments perfused in parallel. The venous partial pressure of nitrogen leaving the l-th body compartment is found by solving the following conservation of mass equation:

$$\frac{{dP}_{vl}^{{N}_{2}}}{dt} = \frac{{Q}_{T} {Q}_{l}}{{λ}_{l} {V}_{tl}} ( {P}_{a}^{{N}_{2}} ( t ) - {P}_{vl}^{{N}_{2}} ( t ) ) , \qquad ( E67 )$$

where Pᵥₗ^{N}^{₂}(t) is the venous (which is assumed to be equal to the tissue) nitrogen partial pressure in body compartment l, [symbol] ₗ is the vascular conductance of body compartment I, λₗ is the blood-tissue partition coefficient of compartment l, Vₜₗ is the tissue volume of compartment l, and Pₐ^{N}^{₂}(t) is the perfusion weighted average arterial partial pressure of N₂ in each alveolar compartment. Pₐ^{N2}(t) is given by

Pₐ^{N}^{₂}(t)=ΣᵢC_{d}_{ᵢ}Fᵢ^{N}^{₂}(t)(P_{b}−P_{H}_{₂}_{O}). (E68)

The mixed venous nitrogen concentration entering the lung is the perfusion weighted concentration leaving the l body compartments,

C_{v̅}^{N}^{₂}(t)=S_{b}ΣₗQₗPᵥₗ^{N}^{₂}(t) (E69)

where S_{b} is the solubility of N₂ in the blood. An analogous approach may be taken with any tracer gases present.

Unlike N₂, O₂ is consumed by the body, and thus if the four compartment model for O₂ were adopted, how this consumption was distributed among the compartments would also need to be determined. As the information with which to do this is not available, for the O₂ stores a single body compartment is assumed. Further, a constant rate of O₂ consumption is assumed, \dot{V}_{O}_{₂}, which is the same as that measured at the mouth during the air breathing period. Thus the governing differential equation for the high O₂ breathing period is

$$\frac{{dC}_{\overline{v}}^{{O}_{2}}}{dt} = \frac{1}{{V}_{L}} ( {Q}_{T} ( {C}_{a}^{{O}_{2}} ( t ) - {C}_{\overline{v}}^{{O}_{2}} ( t ) ) - {\dot{V}}_{{O}_{2}} ) , \qquad ( E70 )$$

where V_{L} is the volume of the compartment and Cₐ^{O}^{₂}(t) is the perfusion weighted average arterial concentration. Cₐ^{O}^{₂} is given by

Cₐ^{O}^{₂}(t)=ΣᵢC_{di}Cₐᵢ^{O}^{₂}(t). (E71)

The O₂ consumption, \dot{V}_{O}_{₂}, is assumed to be equal to that measured at the mouth during the air breathing period, and thus:

$${\dot{V}}_{{O}_{2}} = \frac{1}{{t}_{air}} {\int }_{0}^{{t}_{air}} {F}_{M}^{{O}_{2}} ( t ) u ( t ) dt , \qquad ( E72 )$$

where F_{M}^{O2} is the O₂ fraction measured at the mouth and tₐᵢᵣ is the duration of the air breathing period. This leaves one additional parameter, V_{L}, to be estimated during the parameter recovery procedure.

### Deadspace Compartments

In this section the governing advection equation for a deadspace compartment is derived. It is assumed that the deadspace compartment is a straight cylindrical pipe of radius R and length L. Also it is assumed that gravity can be neglected, and that the flow is: incompressible; independent of z; axial; and independent of the polar co-ordinate θ (i.e. we assume axial symmetry).

Consider a region of axial length dz as illustrated in FIG. 8: The volume of gas g in that region at time t will be

AdzF^{g}(z,t), (E73)

where F^{g} is the fraction of gas g and A is the cross-sectional area of the cylinder. Since diffusion is ignored, the net volume of gas g entering that region at time t is,

u(t)(F^{g}(z,t)−F^{g}(z+dz,t)) (E74)

where u(t) is the measured flow rate. By conservation of mass the change of volume of gas g in this region from t to t+dt is equal to the net volume of gas entering that region over the period and thus

Adz(F^{g}(z,t+dt)−F^{g}(z,t))=dtu(t)(F^{g}(z,t)−F^{g}(z+dz,t)) (E75)

Dividing this expression by dzdt and taking limits as these tend to zero, the governing advection equation for the deadspace compartments is:

$$\frac{\partial {F}^{g} ( z , t )}{\partial t} = - \frac{u ( t )}{A} \frac{\partial {F}^{g} ( z , t )}{\partial z} . \qquad ( E76 )$$

### Parameter Estimation

In this section the minimisation problem that is solved to estimate the parameters of the model (listed in Table 1) from data collected during the washout procedure 100 is defined. For this the washout procedure 100 comprises ten minutes of air breathing followed by six minutes of breathing pure O₂.

The cost function, R, that is minimised to estimate the parameters of the model is

R=Σ_{air-breathing}((F_{M}^{CO}^{₂}(tₗ−F_{S}^{CO}^{₂}(tₗ))²+(F_{M}^{O}^{₂}(tₗ))²+(F_{M}^{O}^{₂}(tₗ)−F_{S}^{O}^{₂}(tₗ))²+Σ_{oxygen breathing} u(tₗ)²(F_{M}^{N}^{₂}(tₗ)−F_{S}^{N}^{₂}(tₗ))², (E77)

where F_{M}^{g} r and F_{S}^{g} are the measured and simulated fractions in the measurement plane of the MFS respectively. The ‘air-breathing period’ for the cost function above is defined as the five minutes of normal breathing immediately preceding the high O₂ breathing phase. The first two minutes of normal breathing are discarded to allow the subject to adjust to breathing on the MFS, and the following three minutes are used to allow the model to settle into a quasi steady state.

In (E77) the CO₂ and O₂ fluxes are considered during the air-breathing period, and the N₂ flux during the high O2 breathing period. The reason for not using N₂ during the air breathing period is that during this period the variation of N₂ is small, and thus the signal is relatively noisy.

During the O₂ breathing phase, we have only an approximate idea of how the venous O₂ and CO₂ concentrations behave. As the N₂ exchange with the blood is relatively limited, the influence of perfusion on its signal is much lower, and thus during the washout only the N₂ flux is considered.

To evaluate the cost function the forward problem must be solved, where the governing equations of the model (E56, E59, E60, E61, E62, E63, E65, E66, E67, E70, E76) are solved with known parameter values. However, when solving the forward problem, as well as values for the parameters and the inspired gas fractions and flows, it is also necessary to know:
a) initial conditions for the differential equations, and
b) the composition of the inspired gas and mixed venous blood.

The initial conditions required are the gas fractions in every deadspace and alveolar compartment, and the starting volumes of the alveolar compartments.

The mixed venous concentrations of O₂ and CO₂ may be estimated using a combination of the model, and data collected from the molecular flow sensor. Although highly damped, these venous concentrations are not entirely constant because volunteers may hyperventilate to some degree when breathing on the molecular flow sensor. For this reason a linear trend is allowed for during the air breathing period. During the air breathing period, a continuous flow version of the model in which all alveolar volumes are set to zero is used to provide initial estimates of the venous CO₂ and O₂ concentrations/partial pressures on every breath from the volumes of these gases exchanged at the mouth. The value of each of these is modelled as a constant value, plus a linear drift over time by:

1. Calculating the breath-by-breath CO₂ and O₂ exchange at the mouth directly from the experimental data.

2. Constructing the continuous flow analogue of the full respiratory model—this is a model in which all alveolar volumes are zero.

3. Using the continuous flow analogue model within a non-linear optimisation routine to estimate, for each breath separately, the mixed venous concentrations/partial pressures and alveolar gas fractions that provide the experimentally measured values for that breath's CO2 and O2 exchange.

4. Employing linear regression to estimate the constant values and linear drift coefficients for the mixed venous values for CO₂ and O₂.

5. Setting the starting alveolar (and connected personal deadspace) gas fractions equal to the values calculated in step 3.

6. Setting the starting volumes of the alveolar compartments by: (i) estimating the difference between the total lung volume at the start of the first breath and FRC from the integral of the flow pattern; and (ii) adding this difference multiplied by the fractional compliance of the compartment to the compartments volume at FRC.

7. Running the full model within a non-linear optimisation routine, such as the function fmincon in Matlab (other non-linear optimisation routines may be used), to fine-tune the constant values for CO₂ and O₂ so as to minimise the squared deviations between experimental data and model output for the breath-to-breath gas exchange data.

Conversions between partial pressures and concentrations for CO₂ and O₂ in blood are obtained using a model of CO₂ and O₂ gas carriage by blood (such as Rees S E, Klæntrup E, Handy J, Andreassen S, Kristensen S R. “Mathematical modelling of the acid-base chemistry and oxygenation of blood: a mass balance, mass action approach including plasma and red blood cells.” Eur J Appl Physiol 108: 483-494, 2010).

During the high O₂ breathing phase, the recirculating arterial O₂ will lead to a rise in the venous O₂ concentrations. A simple model of the body's oxygen stores with a single parameter, V_{L} may be used. To estimate the behaviour of the mixed venous O₂ concentration during the high O₂ breathing period, a value for this parameter must be found. This is achieved by finding the value of V_{L} that ensures the alveolar volume remains stable during the high O₂ breathing phase.

Since the venous concentrations are estimated with the model, they will vary as the parameters of the model change in each cost function evaluation. Thus every time the cost function is evaluated it is necessary to: establish initial conditions; estimate the venous gas concentrations during air breathing; find an appropriate value of V_{L}; and then simulate the full experimental procedure.

The non-linear parameter estimation routine requires initial guesses for all the parameters that are to be estimated. These are set to be standard values, such as those given in Table 2.

Once the non-linear estimation routine completes, the final set of lung parameter values which best fit the experimental data are output. This parameter set, together with dependent variables, provides the quantification of lung function.

Additionally, the pure shunt fraction for the lung (i.e. the difference between the measured and model-predicted SpO₂ values—which measures how much of the blood is not exposed to alveolar gas) may be calculated from the model output for pulmonary venous (systemic arterial) oxygen saturation, the experimentally recorded systemic arterial oxygen saturation and the systemic mixed venous (pulmonary arterial) oxygen saturation.

### Results

Table 2 below gives values for the various parameters, derived values and observed quantities calculated by running the model for a healthy volunteer and which, where needed, may be used as initial conditions for running the optimisation routine for any other subject—the model parameters then being varied in the fitting process to match the particular subject's breathing.

TABLE 2

| Labels | Values | Descriptions |
|---|---|---|
| Patient sex | Male | |
| Patient weight/kg | 78 | |
| Patient height/cm | 177 | |
| Lung parameters | | |
| FRC/l,BPTS | 4.725688443 | Functional residual capacity |
| Equivalent CO2 tissue | 0.25 | Effective tissue vol for CO2 |
| volume | | (fraction of FRC) |
| DS/l,BTPS | 0.225313189 | Deadspace |
| sDS | 0.050065423 | Standardised deadspace (deadspace |
| | | as fraction of FRC) |
| sigma(ln(Cp)) | 0.415668464 | Standard deviation of lung |
| | | compliance distribution (log |
| | | normal) |
| sigma(ln(vCd)) | 0.794680702 | Standard deviation of vascular |
| | | conductance distribution (log |
| | | normal) |
| rho(ln(Cp),ln(vCd)) | 0.8 | Correlation coefficient between log |
| | | lung compliance and log vascular |
| | | conductance distributions |
| sigma(ln(sDS) | 0.309420929 | Standard deviation of standardised |
| | | deadspace distribution (normal) |
| sDs*sigma(ln(sDS) | 0.01549129 | Scaled deadspace distribution width |
| Ventilation | | |
| VI/l/min,STPD | 10.76092456 | Inspired ventilation |
| VE/l/mm,STPD | 10.72092393 | Expired ventilation |
| VA/l/min,STPD | 7.133845402 | Alveolar ventilation (inspired |
| | | ventilaiton minus deadspace |
| | | ventilation) |
| sigma(ln(VA)) | 0.56044194 | Standard deviation of alveolar |
| | | ventilation distribution (log normal) |
| External DS/l | 0.155 | External deadspace (equipment) |
| Blood flow | | |
| Q/l/min | 7.294108949 | Blood flow |
| CvCO2/ml STPD/100 | 49.52947517 | Systemic venous CO2 |
| ml | | concentration |
| CvO2/ml STPD/100 | 15.34975624 | Systemic venous O2 concentration |
| ml | | |
| CO2 Slope/ml | −0.225976651 | Slope of systemic venous CO2 |
| STPD/100 ml min | | concentration |
| O2 Slope/ml | 0.214332972 | Slope of systemic venous O2 |
| STPD/100 ml min | | concentration |
| CaCO2/ml STPD/100 | 44.94896812 | Systemic arterial CO2 |
| ml | | concentration |
| CaO2/ml STPD/100 | 20.27702732 | Systemic arterial O2 concentration |
| ml | | |
| SO2 air/% | 97.2 | Arterial O2 saturation-air |
| | | breathing |
| SO2 air/% | 97.57861506 | Arterial O2 saturation-air |
| | | breathing-model |
| SO2 O2/% | 99 | Arterial O2 saturation-O2 |
| | | breathing |
| SO2 O2/% | 99.93304663 | Arterial O2 saturation-O2 |
| | | breathing-model |
| Shunt fraction/% | 0.390990771 | Shunt Fraction |
| V_L | 16.73387648 | Effective circulating volume of |
| | | distribution for O2 |
| Gas exchange | | |
| VCO2/ml/min | 334.5746455 | CO2 production |
| VO2/ml/min | 364.7054474 | O2 consumption |
| VCO2 Efficiency | 96.49166712 | Efficiency of CO2 production: |
| | | inhomogeneous versus |
| | | homogenous lung |
| VO2 Efficiency | 97.93144679 | Efficiency of O2 consumption: |
| | | inhomogenous versus homogenous |
| | | lung |
| Vdot/Q like parameters | | |
| VA_Q mean | 0.978028359 | Ratio of total alveolar ventilation to |
| | | total blood flow |
| rho(ln(VA),ln(Q)) | 0.776251033 | Corrrelation coefficient between |
| | | log alveolar ventilation and log |
| | | blood flow distributions |
| Ln SD(ventilation) | 0.481803655 | Standard deviation of ventilation- |
| | | perfusion ratio (ventilation) |
| Ln SD(perfusion) | 0.467905015 | Standard deviation of ventilation- |
| | | perfusion ratio (perfusion) |
| Ln SD(volume) | 0.446734952 | Standard deviation of ventilation- |
| | | perfusion ratio (volume) |
| RQ_mean | 0.917383187 | Respiratory quotient |
| sigma(RQ) | 0.315117454 | Standard deviation for respiratory |
| | | quotient between lung units |
| pCO2 pO2 (partial pressures) type | | |
| parameters | | |
| PvCO2/mmHg | 48.40001075 | Systemic venous PCO2 |
| hPvCO2/mmHg | 46.63505599 | Systemic venous PCO2 for |
| | | equivalent homogenous lung |
| (ih-h)PvCO2/mmHg | 1.764954763 | Difference in systemic venous |
| | | PCO2 between inhomogenous and |
| | | homogeneous lung |
| PETCO2/mmHg | 39.95967008 | End-tidal PCO2 |
| hPETCO2/mmHg | 39.32555271 | End-tidal PCO2 for equivalent |
| | | homogenous lung |
| PACO2/mmHg | 39.13710995 | Mean alveolar PCO2 |
| sigma(PACO2)/mmHg | 3.906261331 | Standard deviation for alveolar |
| | | PCO2 between lung units |
| PaCO2/mmHg | 40.89227222 | Systemic arterial PCO2 |
| (a-A)PCO2/mmHg | 1.755162266 | Systemic arterial to alveolar PCO2 |
| | | difference |
| hPACO2/mmHg | 39.30217184 | Alveolar/systemic arterial PCO2 |
| | | for equivalent homogenous lung |
| (ih-h)PaCO2/mmHg | 1.590100376 | Difference in systemic arterial |
| | | PCO2 between inhomogenous and |
| | | homogenous lung |
| PvO2/mmHg | 41.15117784 | Systemic venous PO2 |
| hPvO2/mmHg | 41.13356798 | Systemic venous PO2 for |
| | | equivalent homogenous lung |
| (ih-h)PvO2/mmHg | 0.017609866 | Difference in systemic venous PO2 |
| | | between homogenous and |
| | | inhomogenous lung |
| PETO2/mmHg | 107.5880803 | End-tidal PO2 |
| hPETO2/mmHg | 108.6012316 | End-tidal PO2 for equivalent |
| | | homogenous lung |
| PAO2/mmHg | 108.8288646 | Mean alveolar PO2 |
| sigma(PAO2)/mmHg | 10.18524949 | Standard deviation for alveolar |
| | | PO2 between lung units |
| PaO2/mmHg | 100.1778473 | Systemic arterial PCO2 |
| (a-A)PO2/mmHg | −8.651017259 | Systemic arterial to alveolar PCO2 |
| | | difference |
| hPAO2/mmHg | 108.0606609 | Alveolar/systemic arterial PO2 for |
| | | equivalent homogenous lung |
| (ih-h)PaO2/mmHg | −7.882813554 | Difference in systemic arterial PO2 |
| | | between homogenous and |
| | | inhomogenous lung |

FIG. 5 illustrates the results of performing the method above on a healthy volunteer (left-hand plots A, C and E) and on a volunteer with GOLD stage 2 COPD (right-hand plots B, D and F). FIGS. 5A and 5B show the distributions for ventilation:perfusion ratios

$$( \frac{\dot{V}}{V} )$$

and it can be seen that the variance in FIG. 5B for the COPD volunteer is much greater than for the healthy volunteer in FIG. 5A. FIGS. 5C and D show the compliance:volume ratios

$$( \frac{{C}_{L}}{V} )$$

and FIGS. 5E and F show the vascular conductance:volume ratios

$$( \frac{{C}_{D}}{V} )$$

and again in both cases the variance for the COPD volunteer is much greater than for the healthy volunteer, thus illustrating a much greater heterogeneity (lower homogeneity) for the COPD volunteer's lung.

FIGS. 6A and B show the distributions for the deadspace:volume ratios

$$( \frac{{V}_{D}}{{V}_{A}} )$$

for a healthy volunteer (FIG. 6A) and a COPD volunteer (FIG. 6B), again showing the increase in variance with airway disease.

1. A method of quantifying lung function of a subject comprising the steps of:
a. obtaining plural measurements of the amounts at the mouth of plural respiratory gases in inhaled and exhaled breath as a function of time within each breath of a plurality of successive breaths during a period of steady breathing and a period of inert gas wash-in or wash-out of a respiring subject;
b. outputting the measured amounts to a data processor adapted to model lung function by using a parameterised lung model adapted to predict expired amounts of the plural respiratory gases at the mouth of a respiring subject at the time point of each of said plural measurements;
c. varying the parameters of the lung model to fit the predicted expired respiratory gas amounts to the plural measurements; and
d. outputting at least one parameter of the lung model as a quantifier of lung function.

2. A method according to claim 1 wherein the period of steady breathing and the period of inert gas wash-in or wash-out are simultaneous.

3. A method according to claim 1 wherein the period of steady breathing precedes the period of inert gas wash-in or wash-out.

4. A method according to claim 1 wherein periods of inert gas wash-in alternate with periods of inert gas wash-out.

5. A method according to claim 1 wherein the respiratory gases comprise oxygen, carbon dioxide and an inert gas.

6. A method according to claim 1 comprising estimating, from the measured amounts of respiratory gases, nitrogen wash-out for the respiring subject and varying the parameters of the lung model to fit the predicted expired amount of nitrogen during the wash-out to the estimated nitrogen wash-out.

7. A method according to claim 6 wherein the estimate of the amount of nitrogen is obtained by measuring the amount of water vapour in the breath and calculating therefrom the amount of nitrogen.

8. A method according to claim 1 wherein the measurements of the amounts of respiratory gases comprise measurements of the molar flows, or the total flow with concentrations or fractions of respiratory gases in breath.

9. A method according to claim 1 wherein the measurements of the amounts of respiratory gases are made at least every 50 ms, more preferably at least every 25 ms, more preferably at least every 10 ms.

10. A method according to claim 1 wherein the step of varying the parameters of the lung model to fit the predicted expired respiratory gas amounts to the plural measurements comprises minimizing the sum of the squares of the differences for one or more of the respiratory gases at each expiratory measurement time point.

11. A method according to claim 1 wherein the step of varying the parameters of the lung model to fit the predicted expired respiratory gas amounts to the plural measurements comprises fitting the predicted expired respiratory gas amounts to the expiratory flow profile over each breath of the multiple successive breaths for one or more of the respiratory gases.

12. A method according to claim 1 wherein the step of varying the parameters of the lung model to fit the predicted expired respiratory gas amounts to the plural measurements comprises fitting the predicted expired carbon dioxide and oxygen amounts measured during the period of steady breathing and fitting the inert gas amounts in the period of inert gas wash-in or wash-out.

13. A method according to claim 1 wherein the parameterized lung model models the lung as a plurality of alveolar compartments connected by a respective plurality of personal deadspaces to a common deadspace leading to the mouth.

14. A method according to claim 13 wherein the volumes of the personal deadspaces are distributed with alveolar compartment volume according to a personal deadspace distribution.

15. A method according to claim 13 wherein the parametrized model comprises: the volume of the common deadspace; the fractional volume of each alveolar compartment, being the fraction of the total alveolar volume of each compartment at the functional residual capacity of the model; the fractional expansion or compliance of each alveolar compartment, being approximately the fraction of the flow measured at the mouth received by each alveolar compartment; the volume of the personal deadspace for each alveolar compartment; the vascular conductance of each alveolar compartment, being the fraction of the total perfusion received by each compartment.

16. A method according to claim 13 wherein the model parameters define: a bivariate lognormal distribution for the variation of fractional lung compliance with fractional volume and the variation of vascular conductance with fractional volume, the bivariate lognormal distribution being defined by the variance of the log of the fractional lung compliance with fractional volume distribution, the variance of the log of the vascular conductance with fractional volume distribution, and their correlation.

17. A method according to claim 13 wherein the model parameters define a normal, or lognormal, distribution for the variation of the fractional personal deadspace volume with the fractional compartment volume.

18. A method according to claim 1 wherein measurements of the inspired amounts of respiratory gases are input to the parametrized model.

19. An apparatus for quantifying lung impairment in accordance with the method of claim 1 and comprising:
a molecular flow sensor for obtaining measurements of the amounts at the mouth of plural respiratory gases in inhaled and exhaled breath as a function of time within each breath of a plurality of successive breaths during the period of steady breathing and the period of inert gas wash-in or wash-out of a respiring subject;
a data processor adapted to receive the measurements and to model lung function using the parameterized lung model to predict respiratory gas amounts at the mouth of a respiring subject by varying the parameters of the lung model to fit the predicted expired respiratory gas amounts to the plural measurements;
and adapted to output at least one parameter of the lung model as a quantifier of lung function.

20. Apparatus according to claim 19 wherein the molecular flow sensor is adapted to measure the amounts of oxygen and carbon dioxide in an airway at the mouth of a respiring subject.

21. Apparatus according to claim 19 wherein the molecular flow sensor is adapted to measure the amount of water vapour in an airway at the mouth of a respiring subject and the data processor is adapted to calculate therefrom the amount of inert gas.

22. Apparatus according to claim 19 wherein the molecular flow sensor is adapted to measure the molar flows, or the total flow with concentrations or fractions of respiratory gases in the breath of a respiring subject.

23. Apparatus according to claim 19 wherein the molecular flow sensor is adapted to measure the amounts of respiratory gases at least every 50 ms, more preferably at least every 25 ms, more preferably at least every 10 ms.

24. A computer program comprising program code means for controlling a computer to execute the method of claim 1 by:
a. receiving measurements of the amounts at the mouth of plural respiratory gases in inhaled and exhaled breath as a function of time within each breath of a plurality of successive breaths during a period of steady breathing and a period of inert gas wash-in or wash-out of a respiring subject;
b. modelling lung function by using a parameterised lung model adapted to predict expired amounts of the plural respiratory gases at the mouth of a respiring subject at the time point of each of said plural measurements;
c. varying the parameters of the lung model to fit the predicted expired respiratory gas amounts to the plural measurements; and
d. outputting at least one parameter of the lung model as a quantifier of lung function.

25. The use of a quantified measure of lung inhomogeneity as a biomarker of lung disease wherein the quantified measure is determined by the method of claim 1.

26. The use of a quantified measure of lung inhomogeneity as a biomarker of lung disease according to claim 25 wherein the quantified measure comprises at least one of: a measure of the variation in lung compliance across the lung, a measure of the variation in deadspace fraction across the lung, a measure of the relative inefficiency of oxygen or carbon dioxide exchange between an inhomogeneous and homogeneous lung, and the difference in systemic arterial or mixed venous oxygen or carbon dioxide concentration between a homogenous and inhomogeneous lung.

27. The use of a quantified measure of lung inhomogeneity as a biomarker of lung disease according to claim 26 wherein the measure of the variation in lung compliance across the lung is the standard deviation or variance in the distribution of the log of lung compliance with alveolar volume.

28. The use of a quantified measure of lung inhomogeneity as a biomarker of lung disease according to claim 26 wherein the measure of the variation in deadspace fraction across the lung is the standard deviation or variance of the distribution of deadspace with alveolar volume.
